(19) United States
(12) Patent Application Publication
(10) Pub. No.: US 2020/0397975 A1
(43) Pub. Date: Dec. 24, 2020

(54) BLADDER IRRIGATION TOOL

(71) Applicants: David Kirk, Elk Grove, CA (US); Jeremy Elrod, Elk Grove, CA (US)

(72) Inventors: David Kirk, Elk Grove, CA (US); Jeremy Elrod, Elk Grove, CA (US)

(21) Appl. No.: 16/901,578

(22) Filed: Jun. 15, 2020

Related U.S. Application Data
(60) Provisional application No. 62/863,526, filed on Jun. 19, 2019.

(51) Int. Cl. A61M 3/02; A61M 39/24; A61M 1/00

(52) U.S. Cl. CPC A61M 3/0283; A61M 39/24; A61M 2210/1085; A61M 2205/583; A61M 1/0009

(57) ABSTRACT

A tool is attachable to a bladder irrigation catheter. A body supports multiple fluid pathways between each of the catheter, a supply of irrigation fluid and a waste fluid collector. A syringe or other pressure and vacuum applicator accesses the pathways and powers irrigation flow. A supply port and intake port are connected to a supply pathway leading to the applicator. Similarly, a drain port and discharge port are connected to a drain pathway leading to the applicator. The supply pathway and drain pathway can be selectively blocked so that action of the applicator only drives supply or drain irrigation action. Check valves are located to provide proper fluid flow direction. Bypasses between the supply port and intake port and between the drain port and discharge port can be activated to bypass the supply pathway and drain pathway and allow for gravity flow bladder irrigation through the tool.

### CROSS-REFERENCE TO RELATED APPLICATIONS

This application claims benefit under Title 35, United States Code §119(e) of U.S. Provisional Application Ser. No. 62/863,526 filed on Jun. 19, 2019.

### FIELD OF THE INVENTION

The following invention relates to tools for use in irrigating a patient's bladder. More particularly, this invention relates to tools which attach to a triple lumen catheter or other multi-lumen catheters and also relates to tools for both gravity powered irrigation and forced pressure and suction irrigation of a patient's bladder.

### BACKGROUND OF THE INVENTION

A variety of symptoms and medical conditions suggest that a patient would benefit from bladder irrigation. For instance, when blood clots or other undesirable objects collect within the bladder, irrigation can successfully remove such objects. One known prior art apparatus for bladder irrigation is a triple lumen catheter. Such a catheter has a single proximal end typically fitted with an expandable anchor balloon, to temporarily anchor the proximal end within the bladder. A distal end of the triple lumen catheter includes three separate ports, an inlet port for irrigation fluid, an outlet port for discharge of bladder fluids and an air port for controlling expansion/contraction of the anchor balloon. Three separate lumens within the triple lumen catheter extend along the catheter, one for expansion/collapse of the balloon, and the two others for supply and removal of fluids relative to the bladder.

Generally, supply of fluid into the bladder for irrigation can occur either by gravity forces or by pressurized delivery (or both). Gravity flow can be induced by having irrigation fluid within a container coupled to the input port (also called the solution lumen) elevated above the bladder, so that gravity forces cause irrigation fluid flow into the bladder. Pressurized delivery of irrigation fluids can involve filling of a syringe with bladder irrigation fluid, connecting the syringe to the input port at the proximal end of the triple lumen catheter and expressing the irrigation fluid from the syringe, through the solution lumen of the triple lumen catheter and into the bladder.

Removal of bladder fluid can occur by gravity, pressure assist, or both. For gravity extraction of bladder fluids, the outlet port at the proximal end of the triple lumen catheter is placed below the bladder, so the gravity flow forces drain bladder fluids through the catheter and out of the bladder. For pressure assist, a collapsed syringe can be coupled to the outlet port (also called the outflow lumen) at the proximal end of the triple lumen catheter, and the plunger retracted to generate a vacuum within a chamber of the syringe, and to cause bladder fluid to be forced out of the bladder, into the outflow lumen of the triple lumen catheter and out of the outlet port and into the syringe.

While the triple lumen catheter utilizing known prior art techniques is generally effective, it has numerous drawbacks. As an example, medical personnel must sequentially attach and detach syringes to the various different ports of the triple lumen catheter to perform the various different pressure assist functions. These attachments/detachments are time consuming and present an opportunity for an error to occur. Furthermore, these events each present an opportunity to spill bodily fluids, leading to exposure events for medical personnel, soiling of bed linens and other items, and generally deteriorating the sanitation of the treatment area. If pressure assist is desired both for supply of irrigation fluid into the bladder and for extraction of bladder fluids, and if the same triple lumen catheter is to be utilized, either separate syringes need to be utilized or the syringe must be repeatedly moved between supply and discharge ports of the proximal end of the triple lumen catheter.

Such repeated attachment and detachment of the syringe is magnified when a series of irrigation cycles are desired to be performed. In particular, a syringe is first filled, then detached from a supply container and attached to the inlet port of the triple lumen catheter and is utilized to supply irrigation fluid into the bladder. Next, the syringe is detached from the inlet port and attached to the outlet port of the catheter, and used to extract bladder fluids. Finally, the syringe is detached from the outlet port and attached to a port leading to a bladder fluid collection container. If multiple irrigation cycles are involved, the attachment/detachment cycles are further magnified.

Accordingly, a need exists for a device which can be used with a single triple lumen catheter and a single syringe (or limited number) to provide repeatable bladder irrigation cycles and without requiring repeated attachment/detachment of the syringe or multiple syringes, and which can be used in a simple manner to increase efficiency and accuracy of the bladder irrigation process. Also, a need exists for a manifold which can be used with a triple lumen catheter for bladder irrigation which can operate based on gravity flow and then, at the direction of an appropriate skilled medical practitioner, can be utilized for a pressurized irrigation procedure. Such manifold use would preferably be without requiring adjustment to the triple lumen catheter or gravity flow tubing and reservoirs, both with pressurized assistance for delivery of irrigation fluid and extraction of bladder fluids, or merely for pressurized supply of irrigation fluid and/or pressure assist extraction of bladder fluids.

### SUMMARY OF THE INVENTION

With this invention, a manifold interface medical tool is provided which can be coupled to various proximal ports of a triple lumen catheter to facilitate sanitary, simple and effective bladder irrigation, both of a gravity flow variety and with pressure assist. The triple lumen catheter is not modified in a typical embodiment of this invention. Rather, a proximal end of the triple lumen catheter is placed within and anchored within the bladder. A proximal end of the catheter has liquid ports communicating with an irrigation fluid solution lumen and with a bladder fluid output port. These liquid lumens are accessed by the manifold of this invention.

The manifold is a body formed primarily of rigid material with a series of entry and exit openings and ports associated therewith. In particular, the body includes an intake port for intake of clean irrigation fluid, a supply port for supply of clean irrigation fluid into the solution lumen at the proximal end of the triple lumen catheter, a drain port for removal of bladder fluids from the output lumen of the triple lumen catheter and a discharge port leading to a receptacle for collection of bladder fluids removed from the bladder.

Preferably and to facilitate gravity flow between the various lumens/ports, bypass tubes are preferably provided between the intake port and the supply port and between the drain port and the discharge port. These bypass tubes allow for simple gravity flow through the various openings both into and out of the various lumens at the proximal end of the triple lumen catheter. Gravity forces are generated by providing an irrigation fluid reservoir at the highest elevation, the proximal end of the triple lumen catheter at a middle elevation and with the collection receptacle at a lowest elevation. Preferably these bypass tubes are formed of flexible resilient surgical tubing.

Clips, which can function similar to clothespins, can be placed over these bypass tubes to act as valves to selectively close these bypass tubes. As an alternative, stopcock type valves could be placed upon these bypass tubes, or other forms of valves could be placed on these bypass tubes. When mere gravity flow is desired, clips or other valves would be removed (or placed in an open state) so the gravity flow can occur into the bladder and out of the bladder for irrigation thereof based on gravity forces. These gravity forces could be increased somewhat by elevating the irrigation supply reservoir and lowering the collection receptacle. Further increase of forces causing irrigation of the bladder could be introduced through other portions of the manifold, as further described in detail below.

Each port is also preferably in fluid communication with a corresponding ante-chamber within in the manifold. The ante-chambers which are associated with the supply port and the intake port preferably also access a first chamber through a divider wall. This wall includes holes passing therethrough which allows for flow between the ante-chambers and the first chamber.

These holes are covered with a flexible seal between the first chamber and the intake port and an opposing seal between the supply port and the first chamber. The seals are preferably configured to be formed of a flexible resilient material, such as rubber. Most preferably, a contour of the seal is in the form of a flap with the hole surrounded by a protrusion, and the flap having a corresponding cut out, so that the flap and hole have a complemental shape to provide a tight seal when closed. By having the protrusion taper frusto-conically, and by having the cutout also taper frusto-conically with a similar diameter, thickness and taper angle, the protrusion cutout can form a tight but temporary seal.

The flaps are positioned to have a relaxed state adjacent to the wall and overlying the hole, to cause a closed and liquid tight seal. The seal is enhanced when back flow pressures are encountered pushing the flap of the seal against the wall. However, when reverse pressure forces are applied, tending to push the flap away from the wall, the seal is opened and fluid flow can occur through the hole in the wall, and between the first chamber and the two ante-chambers. The two seals are oriented in reverse orientation so that they act as one way check valves, preventing flow other than into the intake port and out of the supply port. Such flow can occur based on gravity forces through the ante-chambers and the first chamber, or can occur through the bypass tube. The entire manifold is preferably closed off on an upper side by a preferably at least partially clear cover, so that to some extent fluid flow through the manifold can be visually inspected and monitored by a user, as an option.

The first chamber also includes communication with a first tube which leads to a pressure control port in the manifold. A syringe or other pressure and vacuum applicator (e.g. a pump) can be attached to the pressure control port. The syringe can apply pressure or suction/vacuum to the first chamber, by pushing or pulling on a plunger of the syringe. For instance, when the plunger is retracted, clean irrigation fluid is caused to be drawn into the intake port, into the associated ante-chamber, through the flexible seal, into the first chamber, through the first tube, through the pressure control port and into (or at least toward) the syringe.

Within the syringe, the clean irrigation fluid can be inspected to verify that it is clean, and can also be measured. The plunger can then be depressed, causing supply of irrigation fluid from the syringe, through the pressure control port through the first tube, into the first chamber, and then through the opposing seal, into the associated ante-chamber and out through the supply port to the solution lumen at the proximal end of the triple lumen catheter, for delivery into the bladder. Because the flexible seal and opposing seal have opposing orientations, they prevent reverse flow during such a clean fluid loading process. In this way, contaminated fluid within the manifold or within the triple lumen catheter resists travel back into the supply of clean irrigation fluid, and the flow of clean irrigation fluid is directed into the bladder by syringe plunger depression, without the syringe needing to be attached/detached.

A similar arrangement within the manifold is provided with a second chamber communicating with the drain port and discharge port associated with the output lumen at the proximal end of the triple lumen catheter. This second chamber is in fluid communication with a second tube which leads to the pressure control port. A flexible seal and opposing seal are positioned within a divider wall in the body which divides the second chamber from ante-chambers associated with the drain port and the discharge port. Thus, flow through the second chamber is allowed, while reverse flow is prevented. This flow includes flow out of the bladder, through the second chamber and out to a collection receptacle.

Such flow of bladder fluid out of the bladder through the triple lumen catheter can occur by gravity flow or can be pressure assisted by attaching a syringe or other pressure device to the pressure control port. Such attachment of the syringe can be directly to the pressure control port or through tubing or some other fluid conduit between the syringe and the pressure control port. Retraction of a plunger of the syringe causes fluid flow out of the bladder into the second chamber and then through the second tube, and through the pressure control port. This bladder fluid can be inspected while it is in the syringe. Depressing of the syringe then causes bladder fluid to pass from the syringe through the pressure control port, through the second tube, through the second chamber, through the discharge port and into the collection receptacle, which is typically clear and provides a second opportunity for inspection of bladder fluids. All of these steps are performed from start to finish with as few as one syringe and without multiple attach/detach steps.

The first tube and second tube are preferably each flexible resilient surgical tubing or other tubing type structures extending between the first chamber or second chamber on one side and the pressure control port on opposite sides. Preferably a selector knob is provided adjacent to the first tube and the second tube. The selector knob can compress either the first tube or the second tube so that either the first tube or the second tube is closed off. In this way, the pressure control port interacts with either the first chamber or the second chamber, but does not act on both ports simultaneously.

As an optional alternative, a pair of pressure control ports can be provided, one interfacing with the first tube and one interfacing with the second tube. Two separate syringes can be provided, one for handling and pressure assist supply of irrigation fluid and the other for handling and removal of bladder fluids. With such a dual port design, either a single syringe can be moved between the two pressure control ports, or two syringes can be utilized. Such a two syringe solution reduces cross-contamination, especially when multiple cycles of irrigation are to be provided, and limits to only two syringes the number of syringes required. Text is preferably printed on the body which assists in proper connection of ports together and control of the pressure control port and control of the bypass tubes and associated clips.

### OBJECTS OF THE INVENTION

Accordingly, a primary object of the present invention is to provide a bladder irrigation tool for simple powered flushing of irrigation fluid through the bladder of a patient for irrigation thereof.

Another object of the present invention is to provide a bladder irrigation tool which can be powered by a syringe.

Another object of the present invention is to provide a bladder irrigation tool which can both use a syringe to drive irrigation fluid into the bladder and utilize the same syringe for drawing waste fluid out of the bladder.

Another object of the present invention is to provide a bladder irrigation tool which can either be used for forced bladder irrigation or for unforced gravity flow bladder irrigation.

Another object of the present invention is to provide a tool attachable to a triple lumen catheter for providing a high degree of control of the bladder irrigation process for a medical professional.

Another object to the present invention is to provide a bladder irrigation tool which simplifies the process of flushing the bladder of undesirable waste.

Another object to the present invention is to provide a bladder irrigation catheter which is simple to install on a multi-lumen catheter.

Another object of the present invention is to provide a method for irrigating a bladder of a patient which is simple to practice and provides a high degree of control over the irrigation process.

Other further objects of the present invention will become apparent from a careful reading of the included drawing figures, the claims and detailed description of the invention.

### BRIEF DESCRIPTION OF THE DRAWINGS

FIG. 1 is a schematic of a known prior art triple lumen catheter and known prior systems for bladder irrigation, of which the method and tool of this invention are provided as an improvement.

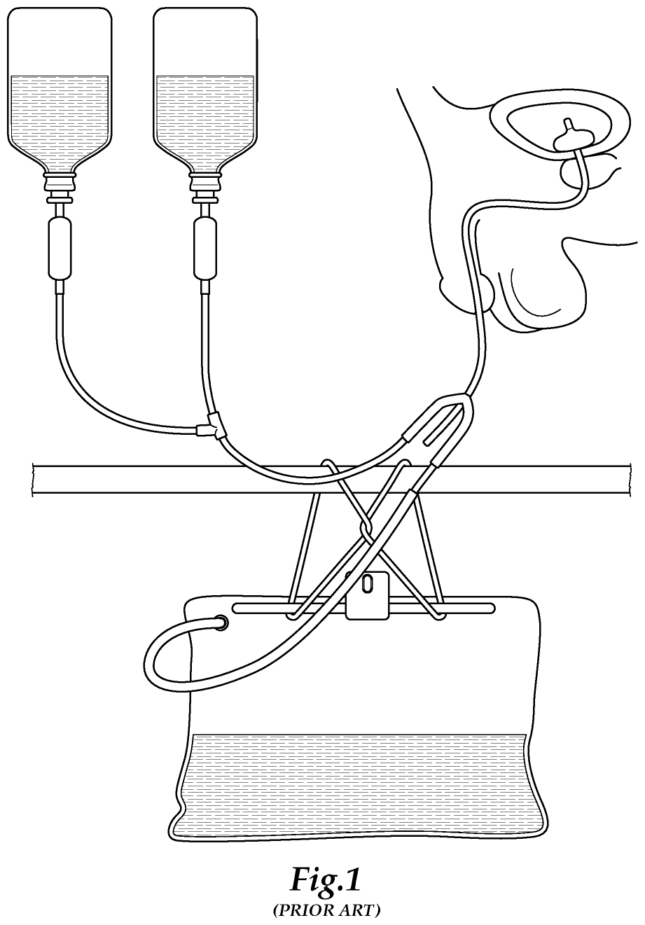

FIG. 2 is an exploded parts view of the bladder irrigation tool of this invention which is connectable to a triple lumen catheter or other multi-lumen catheter, and coupleable to a syringe or other pressure and vacuum applicator.

FIG. 3 is a side perspective exploded parts view of that which is shown in FIG. 2.

FIG. 4 is a top plan view of the irrigation tool of this invention coupled to a triple lumen catheter and to a syringe, and with a cover thereof removed to show interior details of the tool.

FIG. 5 is a plan view similar to that which is shown in FIG. 4, and in a first step of drawing irrigation fluid from a source of irrigation fluid in a first step. Moving irrigation fluid is shown with light stipple shading.

FIG. 6 is a plan view similar to that which is shown in FIG. 5, but in a second step of advancing the irrigation fluid from the syringe to the bladder through the solution lumen of the triple lumen catheter. Moving irrigation fluid is shown with light stipple shading.

FIG. 7 is a plan view similar to that which is shown in FIG. 6, but in a third step of drawing waste fluid out of the bladder along the output lumen of the triple lumen catheter and into the syringe. Moving waste fluid is shown with dark stipple shading.

FIG. 8 is a plan view similar to that which is shown in FIG. 7, but in a fourth step of advancing the waste fluid from the syringe to the waste fluid collector through the discharge port. Moving waste fluid is shown with dark stipple shading.

FIG. 9 is a top plan view similar to that which is shown in FIGS. 4-8, but with the clips on the bypass lines removed and with the tool in a gravity irrigation continuous flow mode. Moving irrigation fluid is shown with light stipple shading and moving waste fluid is shown with dark stipple shading.

### DESCRIPTION OF THE PREFERRED EMBODIMENT

Referring to the drawings, wherein like reference numerals represent like parts throughout the various drawing figures, reference numeral 10 is directed to a tool for assisting in bladder irrigation. A multi-lumen catheter, such as a triple lumen catheter T is placed extending into the bladder of a patient. The triple lumen catheter T includes a solution lumen S into which clean irrigation fluid is supplied, and an outflow lumen O from which waste fluid is drawn. The tool 10 has ports thereon which connect to these lumens S, O of the triple lumen catheter T, as well as to an irrigation fluid source and a waste fluid collector. The tool 10 can both drive, such as with a syringe Y, flow of irrigation fluid and waste fluid in a first mode of operation, and facilitate natural gravity flow irrigation of the bladder in a second mode of operation.

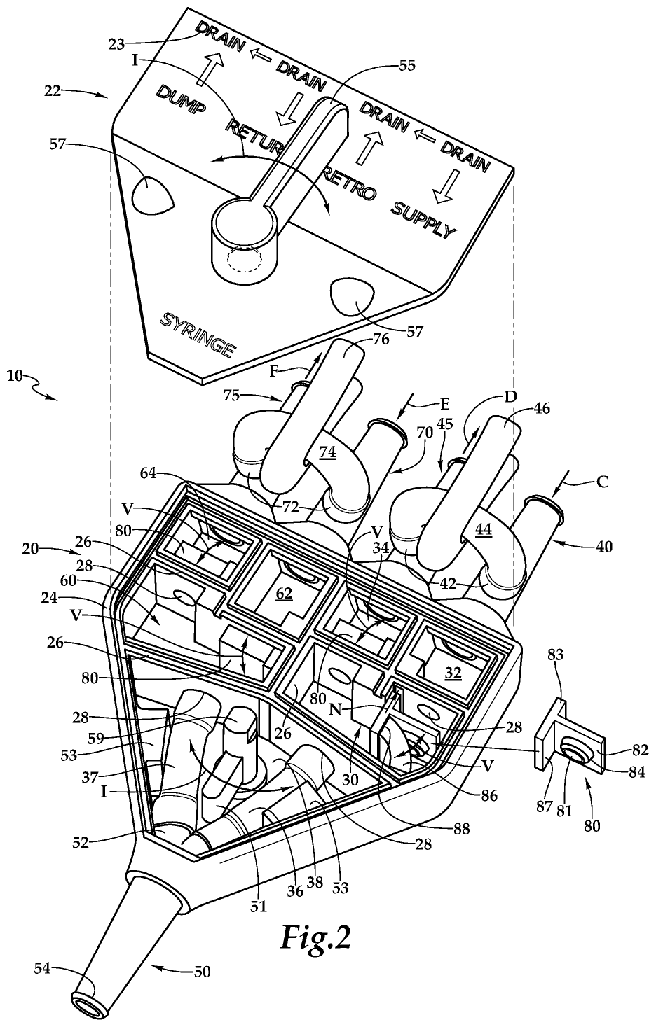

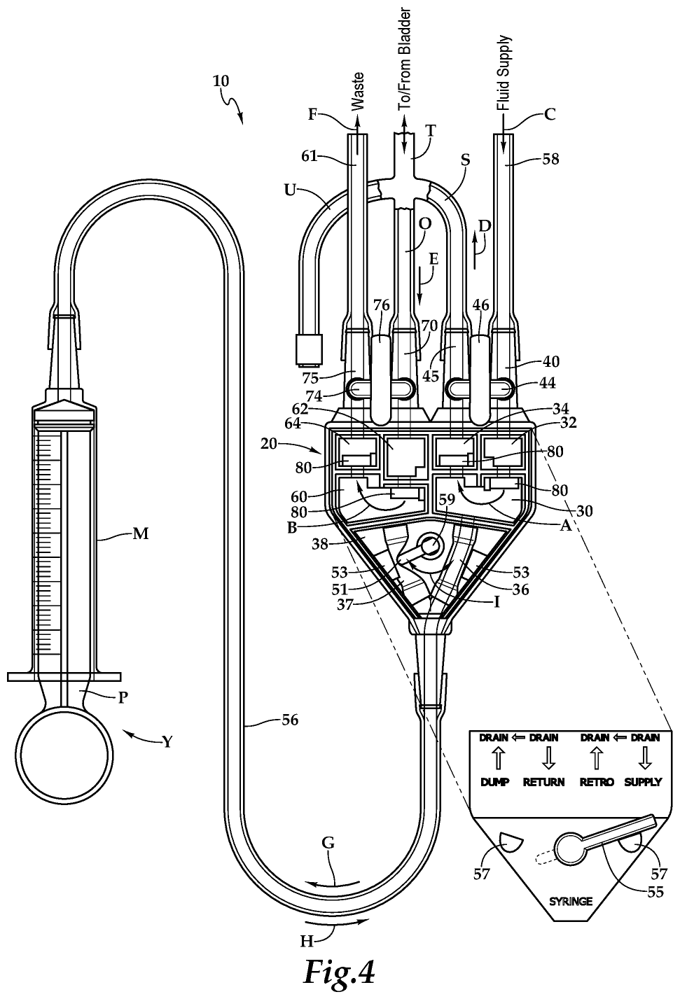

In essence, and with particular reference to FIGS. 2-4, basic details of this invention are described, according to one embodiment. The bladder irrigation tool 10 is housed within a body 20 which is configured as a manifold chamber having pathways therein joining various different ports thereof. A first chamber 30 within the body 20 defines part of a supply pathway with an intake port 40 and a supply port 45. This first chamber 30 is also in fluid communication with a pressure control port 50. A second chamber 60 within the body 20 defines at least part of a drain pathway with a drain port 70 and a discharge port 75. This second chamber 60 also includes communication with the pressure control port 50. A pathway selector selectively blocks either the supply pathway or the drain pathway from being open to the pressure control port 50, leading to a syringe Y (FIG. 4) or other pressure and vacuum applicator coupled to the pressure control port 50, to draw a vacuum or add pressure to either the supply pathway or the drain pathway, depending on the status of the pathway selector. Check valves 80 are strategically placed along the supply pathway and the drain pathway so that the intake port 40 only allows flow into the supply pathway toward the pressure control port 50, and the supply port 45 only allows flow out of the supply pathway away from the pressure control port 50. The drain port 70 only allows flow into the drain pathway toward the pressure control part 50 and the discharge port 75 only allows flow out of the drain pathway away from the pressure control port 50. A triple lumen catheter T or other multi-lumen catheter can be placed accessing a patient's bladder and with a solution lumen S coupled to the supply port 45, the outflow lumen 0 being coupled to the drain port 70, intake port 40 coupled to a source of irrigation fluid, and the discharge port 75 coupled to a waste fluid collector. Positioning of the pathway selector and action of the syringe Y allows for forced irrigation of the patient's bladder. A first bypass tube 44 and a second bypass tube 74 allow for short circuiting the intake port 40 to the supply port 45 and short circuiting of the drain port 70 to the discharge port 75, to facilitate gravity induced bladder irrigation without forced pressure and vacuum, when the bypass tubes 44, 74 are opened.

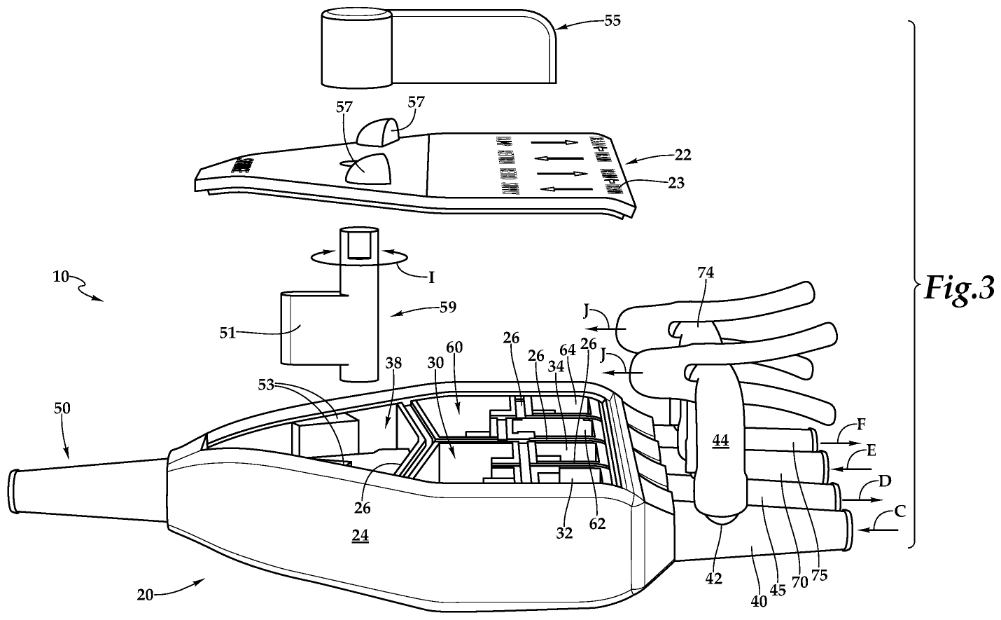

More specifically, and with particular reference to FIGS. 2 and 3, finer details of the body 20 of the tool 10 are described according to this disclosed embodiment. The body 20 is an enclosure primarily of fixed walls dividing separate compartments from each other, and with holes 28 strategically placed through various walls to provide access between certain ones of the compartments within the body 20. While in this embodiment the body 20 is primarily configured with separate compartments and holes in walls dividing the compartments, at least some fluid containing structures are in the form of tubes, and especially tubes having flexible walls for containing fluids within the body 20. Alternatively, at least some of the compartments within the body 20 could be replaced with tubes, or various tubes with the body 20 could be replaced with compartments or other fluid holding regions. Terms such as “port” and “pathway” define fluid spaces which could be compartments, tubes, or other spaces in which liquid can reside and flow.

The body 20 preferably is formed of two main parts with lower portions defined by the body 20, and with a cover 22 being generally planar and overlying the lower portion of the body 20, to provide an enclosed space other than where ports provide access into and out of compartments within the body 20 of the tool 10. The body 20 and cover 22 are preferably formed of an injection multiple plastic, which also is a plastic which is generally biocompatible in that it is inert to fluids which might be passed into or drawn out of the body of a patient, so that the tool 10 is generally biocompatible. As alternatives to injection molded manufacture, the body 20 and cover 22 could be formed by machining, stamping, additive manufacturing, or other manufacturing techniques, including assembly from separate sub-compartments and sub-components either bonded together with an adhesive, welded, or coupled together with fasteners.

The tool 10 can, in one embodiment, be configured to allow for disassembly and cleaning, such as with an autoclave or other sterilization device, to allow for reuse. In other embodiments, the tool 10 would be optimized for low cost manufacture and be single use and disposable. Elements used with the tool 10 and in some embodiments included as part of the tool 10 can include a triple lumen catheter T known in the prior art, a syringe Y known in the prior art, and various different liquid transport tubes and lines to connect various ports of the tool 10 to other medical fluid handling devices. While this disclosed embodiment shows use of the tool 10 with a triple lumen catheter T, other multi-lumen catheters, or conceivably multiple separate catheters routed separate from each other into the bladder of a patient, could be utilized in alternative embodiments.

The cover 22 preferably has a perimeter shape which matches a perimeter shape of the lower portion of the body 20 so the cover 22 completely encloses an interior of the body 20. The cover 22 in one embodiment is at least partially transparent so that fluid flow within the body 20 can to some extent to be viewed. The cover 22 preferably includes indicia 23 on a surface thereof which provides instruction as to which of the ports on the body 20 are used for which purposes, to facilitate proper interconnecting of the ports to other lines and tubes for proper utilization of the tool 10. This indicia 23 can also work together with the pathway selector which has a selector shaft 59 which preferably extends axially up through the cover 22, so that the pathway selector can be properly positioned during operation of the tool 10. The cover 22 includes a hole through which the pathway selector shaft 59 (FIGS. 2 and 3) can pass, and with a selector knob 55 at an upper end of the selector shaft 59. Stops 57 are built into the cover 20 to prevent the selector knob 55 from rotating too far (along arrow I of FIGS. 2 and 3).

The body 20 is divided into compartments by including outer walls 24 defining a perimeter of the body 20 and divider walls 26 inboard of the outer walls 24. Some of these divider walls 26 include holes 28 passing therethrough. These holes 28 thus join compartments together to form pathways, such as the supply pathway and the drain pathway, made up of multiple separate compartments in this embodiment. Each of the walls 24, 26 preferably extend up from a generally planar floor to a common height within an upper plane. A lower surface of the cover 22 is preferably substantially planar and abuts against upper edges of these walls 24, 26. O-rings, adhesive bonding material, sonic welding, heat welding, or other sealing structures or techniques seal the undersurface of the cover 22 to these walls 24, 26 so that compartments within the body 20 avoid any fluid leakage therebetween, other than when passing through the holes 28 and along the defined pathways for operation of the tool 10.

A first chamber 30 defines a manifold space within the supply pathway. This first chamber 30 is a larger compartment than other compartments within the supply pathway in this particular embodiment. Other portions of the supply pathway include a fore chamber 32 between the first chamber 30 and an intake port 40, and an aft chamber 34 between the first chamber 30 and a supply port 45. Divider walls 26 between the first chamber 30 and the fore chamber 32 and between the first chamber 30 and the aft chamber 34 include holes 28 thereon, so that the supply pathway has fluid communication from the intake port 40, through the fore chamber 32, through the first chamber 30, through the aft chamber 34 to the supply port 45.

Furthermore, this supply pathway is accessed by a first tube 36 passing from the first chamber 30 to the pressure control port 50. This first tube 36 is a hollow cylindrical section of flexible wall tubing in this embodiment, to facilitate it being closed off by the pathway selector under control of an operator, so that access between the first chamber 30 and the pressure control port 50 (and hence access to the syringe Y or other pressure and vacuum applicator) can be selectively shut off by the pathway selector. While this first tube 36 is disclosed in this embodiment as such a flexible wall tube, the first tube 36 could alternatively be provided as a compartment or other fluid containing space which can be selectively opened or closed for access therethrough by fluids.

Check valves 80 are positioned adjacent to the holes 28 between the fore chamber 32 and first chamber 30 and between the aft chamber 34 and first chamber 30. These check valves 80 are oriented to limit flow through the holes 28 to flow in a single direction (along arrow A of FIG. 4). In particular, the check valve 80 between the fore chamber 32 and the first chamber 30 is oriented to allow flow into the first chamber 30 from the intake port 40, and to prevent flow from the first chamber 30 and out of the intake port 40. The check valve 80 between the aft chamber 34 and the first chamber 30 is configured to allow flow from the first chamber 30 to the aft chamber 34 and out of the supply port 45, and to prevent flow from the supply port 45 through the aft chamber 34 and into the first chamber 30. In this embodiment, no check valve is provided at the junction between the first chamber 30 in the first tube 36 leading to the pressure control port 50. Rather, the syringe Y or other pressure and vacuum applicator can draw fluid from the intake port 40, through the first chamber 30, along the first tube 36, through the pressure control port 50 and into the syringe Y (or at least a line leading to the syringe Y). Then, when the syringe Y has its plunger P depressed, fluid is driven from the chamber of the syringe Y, along the line 56, through the pressure control port 50, along the first tube 36, into the first chamber 30, and then through the aft chamber 34 and through the supply port 45.

Each check valve 80 preferably includes a seat 82 of rigid form having an orifice 81 passing through and generally aligned with one of the holes 28 passing through one of the divider walls 26 adjacent to the first chamber 30. The seat 82 preferably includes a tab 83 which can be received within a cavity 85 for securing of the seat 82 the orifice 81 aligned with the hole 28 which is to be limited to one way flow therethrough. The seat 82 could otherwise be bonded or otherwise attached to the divider wall 26 or other adjacent structures to properly align the orifice 81 of the seat 82 with the hole 28. As a further alternative, the orifice 81 could merely be formed in the divider wall 26 surrounding the holes 28 so that the divider wall 26 itself would be the seat 82 with the orifice 81 being the hole 28 passing through the divider wall 26. By providing a cavity 85 in a portion of the divider walls 26 which can receive a portion of the seat 82, and with the orifice 81 aligned with the hole 26, the tab 83 can be placed into the cavity 85 (along arrow N of FIG. 2) to allow for removable attachment of each check valve 80 assembly. Such removal could be convenient during initial construction of the tool 10, and also could facilitate replacement should the tool 10 be made to be modular and refurbished/sanitized for repair and/or reuse.

A collar 84 on the seat 82 surrounds the orifice 81 and preferably extends away from the surface of the seat 82 surrounding the orifice 81 in a tapering fashion so that a tip of the collar 84 has a lesser diameter and a root of the collar 84 adjacent to the seat 82. A flap 86 formed of resilient material is preferably connected at a root 88 to an extension 87 extending from the seat 82 or otherwise anchored to a divider wall 26 or outer wall 24 or other structure within the body 20. This flap 86 preferably has a recessed cavity shaped to match a size and shape of the collar 84, so that when the flap 86 is closed, the collar 84 extends into this cavity and a tight fit is provided between the resilient material formula flap 86 and the collar 84. By making a perimeter of this recess having a frusto-conical tapering form matching a frusto-conical tapering form of the collar 84, secure alignment and tight ceiling of the flap 86 is provided.

The flap 86 preferably is biased toward a closed position. However, when a vacuum is pulled on a compartment on a side of the flap 86 opposite the seat 82, the flap 86 is pulled off of the seat 82 and the orifice 81 of the check valve 80 is opened for fluid flow through orifice 81 and generally toward and around the open flap 86. When pressure is restored on the side of the flap 86 opposite the seat 82, the flap 86 is forced closed against the seat 82. The resilient nature of the flap 86 causes a tight seal between the flap 86 and seat 82 and check valve 80 is thus closed and prevents flow through the orifice 81. While the various check valves 80 can be of a slightly different form, they generally function in the same manner, and are strategically positioned to allow or prevent flow of fluids in a desired or undesired direction, so that operation of the tool occurs according to design, such as that disclosed herein.

The pressure control port 50 is coupled to the first tube 36 leading to the first chamber 30, and also is connected to a second tube 37 leading to a second chamber 60 associated with a drain pathway within the body 20 of the tool 10. The first tube 36 and second tube 37 come together at a dual path end 52 of the pressure control port 50. Thus, flow into and out of the pressure control port 50 on an interior side of the body 20 can occur into and out of either the first tube 36 or the second tube 37. The pressure control port 50 has a single path end 54 opposite the dual path end 52 on the side of the pressure control port 50 most distant from an interior of the body 20. This single path end 54 is coupled to some form of pressure and vacuum applicator. In the embodiment disclosed, this applicator is a syringe Y coupled through a line 56 to the single path end 54 of the pressure control port 50. This line 56 can be flexible or rigid. In one embodiment, the syringe Y could be coupled directly to the single path end 54 of the pressure control port 50.

The first tube 36 and second tube 37 are placed alongside each other within a selector chamber 38 adjacent to the pressure control port 50. Each of these tubes 36, 37 are preferably flexible wall resilient tubes which have a relaxed open orientation, but which can be closed off by pinching closed if desired. The pathway selector is configured to pinch off and close the first tube 36 or the second tube 37 (or can be in a neutral position not closing off either of the tubes 36, 37).

The pathway selector includes the selector shaft 59 pivotably supported at a floor of the body 20 within the selector chamber 38 and between the first tube 36 and second tube 37. A finger 51 extends laterally from the selector shaft 59. Reference surfaces 53 are provided on sides of the first tube 36 and second tube 37 which are opposite the selector shaft 39 and finger 51. The finger 51 is long enough that when the selector shaft 59 is rotated (along arrow I of FIGS. 2 and 3) about a pivot axis, the finger 51, which is rigid along with the rigid selector shaft 59, rotates into abutment against one of the tubes 36, 37 and pinches off one of the tubes 36, 37, depending on which direction the selector shaft 59 is rotated.

An upper end of the selector shaft 59 includes a selector knob 55 thereon which is above the cover 22 of the body 20. The selector knob 55 can be rotated (along with the selector shaft 59) to one of two stops 57, at which point the finger 51 is impinging against and shutting off flow through one of the tubes 36, 37. Preferably, the selector knob 55 is intuitively oriented so that when the selector knob 55 points generally toward the intake port 40 and supply port 45, the supply pathway is open and active. At the same time, the second tube 37 is impinged and closed by the finger 51 pressing against the second tube 37 and against the reference surface 53 adjacent to the second tube 37, so that the drain pathway is inactive. Similarly, when the selector knob 55 is pointed generally toward the drain port 70 and discharge port 75, the second tube 37 is open and the drain pathways active, while the finger 51 pinches against the first tube 36 and presses it against the reference surface 53 to close the first tube 36 and to make the supply pathway inactive. In this way, the pathway selector selectively blocks either the supply pathway or the drain pathway.

In other embodiments, rather than utilizing tubes 36, 37 and the finger 51 on the selector shaft 59, the selector shaft 59 could be aligned with a hub having fluid pathways therein which becomes selectively aligned and misaligned with ports in a compartment adjacent to the first chamber 30 and second chamber 40, so the rotation of the hub will cause different fluid lines/pathways to be made active/inactive, in match the same way that stopcocks are configured.

While the pathway selector has a rotating selector shaft 59 and associated selector knob 55, the pathway selector could instead be a sliding lever which might slide to the left or to the right to selectively make active and inactive the supply pathway or the drain pathway. Other pathway selectors could include push buttons, toggle switches, two separate switches instead of a single switch, and other analogous control configurations. Most preferably, these control devices for the pathway selector are manually adjustable to configure the tool 10 for operation as desired.

As an alternative, the pathway selector could be automated. In one form, such an automated pathway selector could include a timer which would time the toggling of the pathway selector between different positions including active for both the supply pathway and the drain pathway, active for only the supply pathway, active for only the drain pathway, and conceivably also in certain embodiments inactive for both the supply pathway and the drain pathway. As an alternative or in addition to such a timer, a computer, such as a processor and a programmable memory or firmware instructions or the like could be programmed or controlled and activate rotational or linear transducers or other devices to interpret digital control signals into physical movement of the pathway selector, such as according to a programmed sequence. A pump could replace the syringe and be controlled by the processor in such an automated alternative.

The intake port 40, supply port 45, drain port 70 and discharge port 75 preferably are all oriented parallel to each other and extending from an end of the body 20 opposite the pressure control port. At a minimum, preferably the supply port 45 and drain port 70 are preferably so oriented together on a side of the tool 10, for convenient attachment to the various lumens of the triple lumen catheter T, for supply of irrigation fluid to the bladder and removal of waste fluid from the bladder. By conveniently placing all of the ports 40, 45, 70, 75 adjacent to each other and on a common side of the tool 10, bypass ports 42 on the intake port 40 and supply port 45 can connect to ends of a first bypass tube 44 without complex additional plumbing. In one embodiment, the first bypass tube 44 is a U-shaped flexible tubular conduit sized to fit tightly over the bypass ports 42 in the intake port 40 and the supply port 45.

The first clip 46, like a clothes pin, is placed on the first bypass tube 44 to shut off the bypass tube 44 and deactivate a bypass mode. When this first clip 46 is removed, fluid flow can occur along the first bypass tube 44 through the bypass ports 42 and between the intake port 40 and supply port 45. This allows for gravity flow delivery of irrigation fluid from an irrigation fluid supply, into the intake port 40, along the bypass tube 44, into the supply port 45 and then into the solution lumen S of the triple lumen catheter T and into the bladder of the patient. The irrigation fluid source would be elevated above the tool 10 and with the tool 10 above (or near an elevation of) the bladder so that gravity flow would induce this flow of the irrigation fluid into the bladder. The first clip 46 can be placed back upon the first bypass to 44, and the syringe Y or other pressure and vacuum applicator can be cycled to draw fluid under suction from the irrigation fluid source toward the syringe Y, and then by cycling into a pressure mode with the syringe Y or other pressure and vacuum applicator, push this irrigation fluid through the intake port 40 and into the solution lumen S of the triple lumen catheter T and into the patient's bladder. Thus, two modes of operation are simply provided during such irrigation fluid supply utilization portion of the bladder irrigation process. The clip 46 could be replaced with a stopcock, and the bypass tube could be rigid.

The body 20 also includes the drain pathway comprised of a second chamber 60 and an associated pre-chamber 62 and post chamber 64, as well as the second tube 37 leading to the pressure control port 50. The pre-chamber 62 is adjacent to the drain port 70 and the post chamber 64 is adjacent to the discharge port 75. The drain pathway is functionally and structurally equivalent to the supply pathway in form and operation within the same interior of the body 20, but located laterally thereto. In this embodiment, the drain pathway is consolidated at a left side of the body 20 with the supply pathway consolidated at a right side of the body 20, when considering the pressure control port 50 and syringe Y to be at a proximal side of the tool 10, and with the ports 40, 45, 70, 75 of the body 20 at a distal end of the tool 10.

The second chamber 60 and pre-chamber 62 and post chamber 64 are separate compartments within the body 20 and have holes 28 in divider walls 26 similar to those described above with respect to the supply pathway. Furthermore, check valves 80 are placed adjacent to these holes 28 and with these check valves 80 generally matching the form and operation of the check valves 80 described above. Check valves 80 are oriented so that fluid flow is allowed from the pre-chamber 62 and drain port 70 into the second chamber 60, and preventing flow from the second chamber 60 through the pre-chamber 62 into the drain port 70. A check valve 80 is also placed between the post chamber 64 in the second chamber 60 so that fluid flow can occur from the second chamber 60 to the post chamber 64, but is prevented from occurring from the post chamber 64 to the second chamber 60. Fluid flow through the second chamber 60 thus generally occurs along arrow B of FIG. 4. No check valve is provided adjacent to the second tube 37. Rather, control of the flow through the second tube 37 is provided by adjustment of the pathway selector described in detail above. A second bypass tube 74 and second clip 76 are located between the drain port 70 and the discharge port 75 for gravity flow bladder drainage to a lowest elevation collection receptacle.

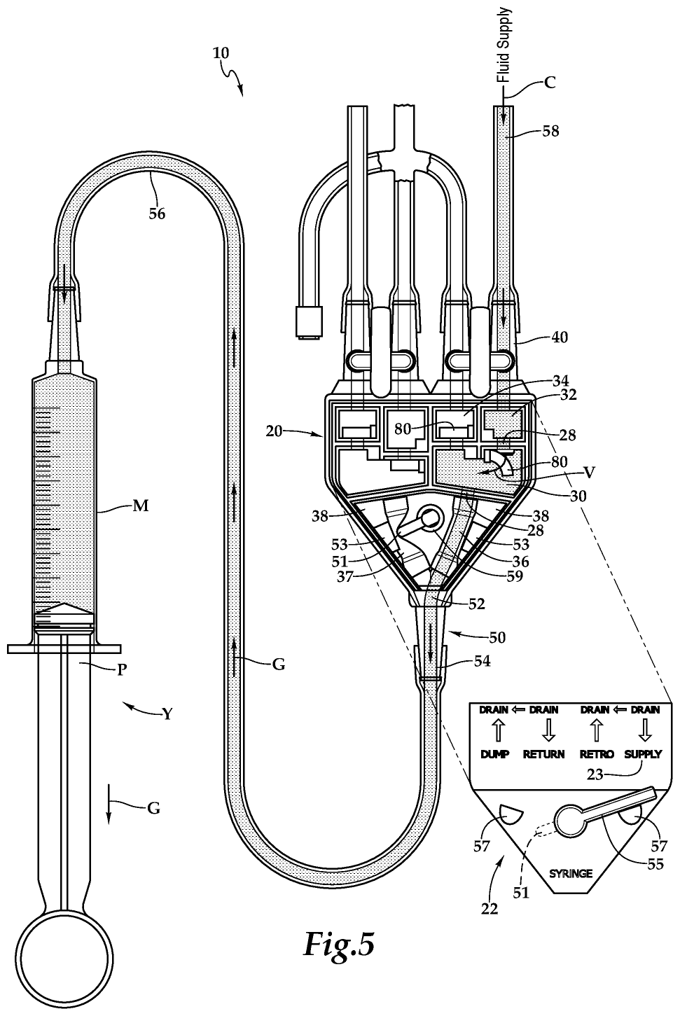

In use and operation, and with reference to FIGS. 5-9, details of the operation of the tool 10 are described according to a first mode where flow is induced for bladder irrigation, and according to a second mode where flow occurs mainly by gravity forces.

With particular reference to FIG. 5, a first step in the first mode of operation is to draw fresh irrigation liquid from a source of irrigation fluid along a liquid supply line 58. This fluid supply line 58 is connected to the intake port 40. A plunger P of the syringe Y is retracted (along arrow G) to draw a vacuum on the first chamber 30. In one embodiment, a syringe Y and flex line 56 between the syringe Y and the pressure control port 50 is first loaded with liquid so that as much of the supply pathway is filled with liquid as possible, and to avoid incompressibility issues associated with too much air in any of the pathways.

The plunger P is drawn back (along arrow G of FIG. 5) until the desired amount of irrigation fluid has been drawn at least into the first chamber 30 (along arrow C), and typically also into the line 56 and into the chamber M of the syringe Y. If desired, an amount of fluid which has been drawn into the tool 10 and syringe Y in this first step can be measured. This first step occurs while the first clip 46 is in place on the first bypass tube 44 to prevent bypass flow, and with the selector knob 55 oriented generally pointing toward the intake port 40 and supply port 45, and with the second tube 37 impinged and closed by the finger 51 of the selector shaft 59.

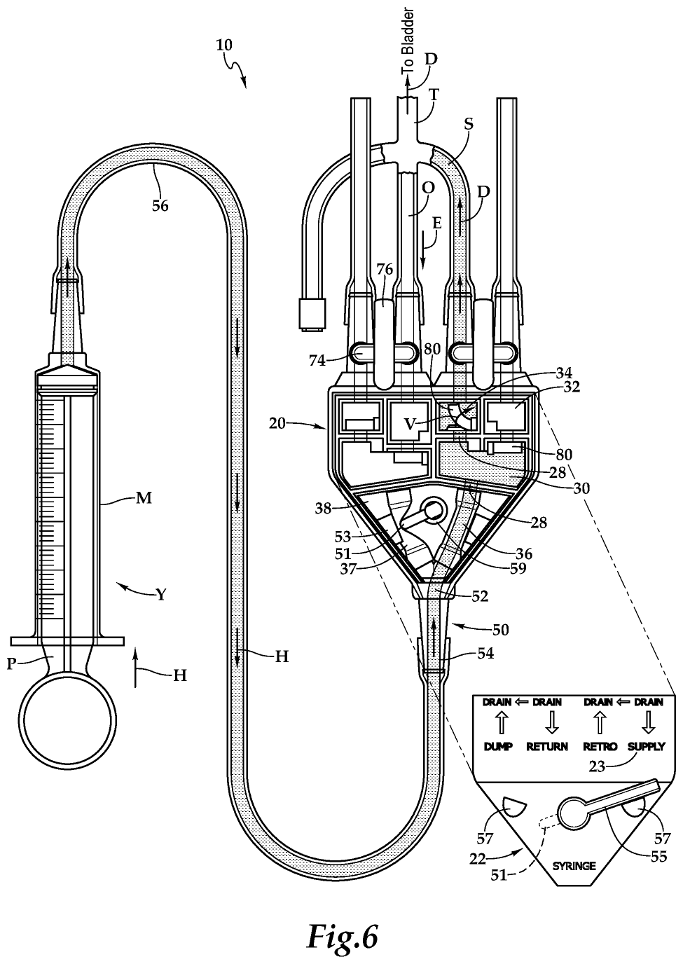

In a second step depicted in FIG. 6, the plunger P of the syringe Y is depressed (along arrow H of FIG. 6) to cause the irrigation fluid to be pressurized and advanced along arrow H into the pressure control port 50, along the first tube 36, and then to the first chamber 30. This pressurized fluid then passes into the aft chamber 34 when the check valve 80 opens under these pressure forces (along arrow D of FIG. 6) and then flows through the supply port 45 and along arrow D into the solution lumen S of the triple lumen catheter T and into the bladder. The second step also occurs with the first clip 56 in place closing off the first bypass tube 44 and with the pathway selector configured with the selector knob 55 pointing generally toward the intake port 40 and supply port 45, and with the second tube 37 closed off by the finger 51 of the pathway selector.

If desired, the first step and second step can be repeated multiple times, such as when a smaller syringe Y is utilized, or if the bladder of the patient is larger, or based on other therapeutic criteria. In one embodiment, the irrigation fluid can be allowed to move under gravity forces out of the bladder over time, by flow along the outflow lumen O of the triple lumen catheter T as waste fluid, passing along arrow E at least back from the bladder, and potentially to a waste fluid collector without waste ever passing through the tool 10, when so used.

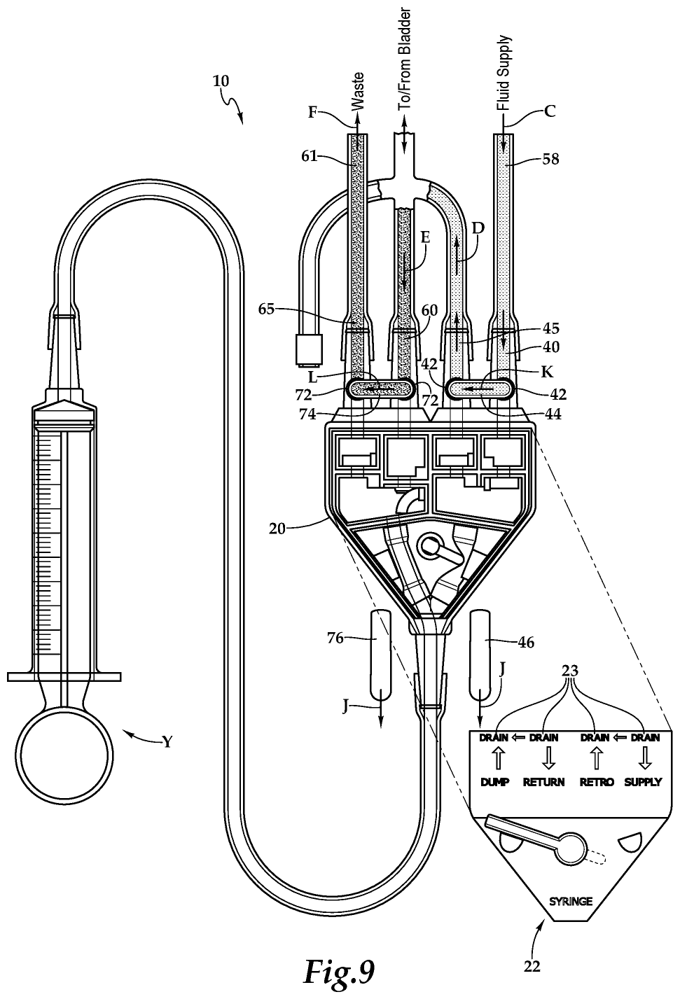

As a slight variation, the irrigation fluid source could be elevated and the first clip 46 removed (along arrow J of FIG. 9) from the first bypass tube 44, and irrigation fluid from irrigation fluid source could be allowed to flow by gravity along arrow C into the intake port 40, then through the bypass ports 42 and the bypass tube 44 to the supply port 45 and into the triple lumen catheter T and into the bladder, and then drain out on the outflow lumen O of the triple lumen catheter T to a waste fluid collector or other discharge site. Thus, in one embodiment, the tool 10 can be utilized with only the supply pathway, either in an active mode where flow is induced or in a passive mode where flow occurs by gravity forces.

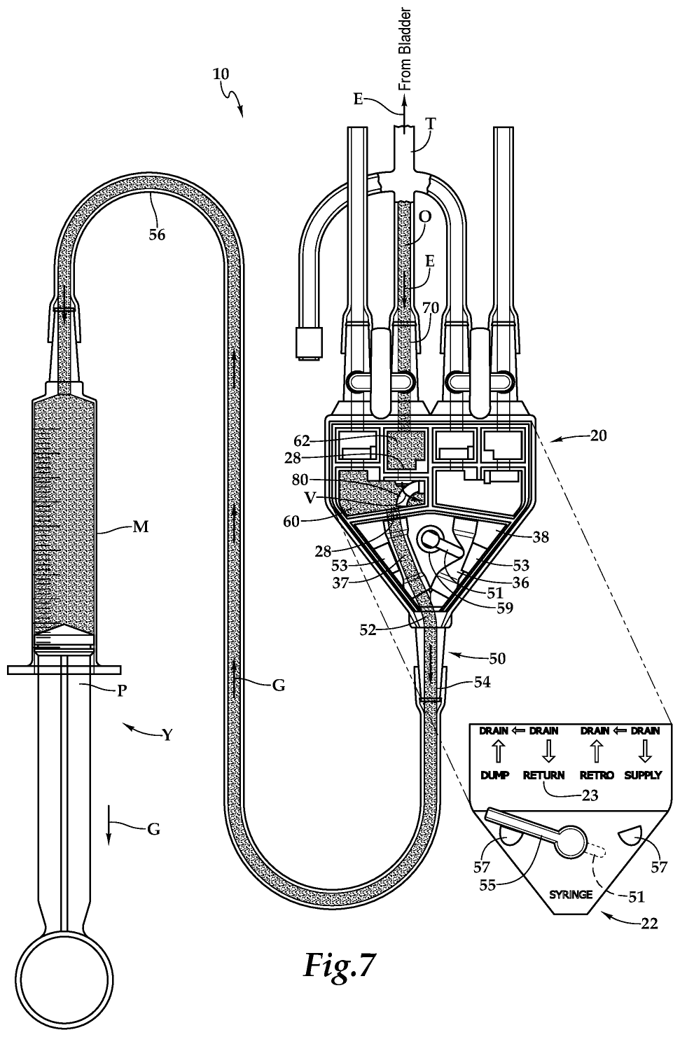

For most active inducement, and according to a third step disclosed in FIG. 7, the pathway selector is rotated (along arrow I of FIGS. 2-4) so that the first tube 36 is closed off and the second tube 37 is opened. The plunger P of the syringe Y is retracted (along arrow G), causing waste fluid to be drawn along arrow G into the chamber M of the syringe Y from the pressure control port 50 and through the second tube 37 and second chamber 60, after flowing through the pre-chamber 62 and drain port 70 coupled to the outflow lumen 0 of the triple lumen catheter T. Waste fluid from the bladder thus passes, along arrow E, through the drain port 70, and at least as far as the second chamber 60, and typically beyond and toward or into the chamber M of the syringe Y. During the third step, the second clip 76 is preferably in place on the second bypass tube 74 so that the bypass port 72 on the drain port 70 and discharge port 75 are not active, but rather closed off.

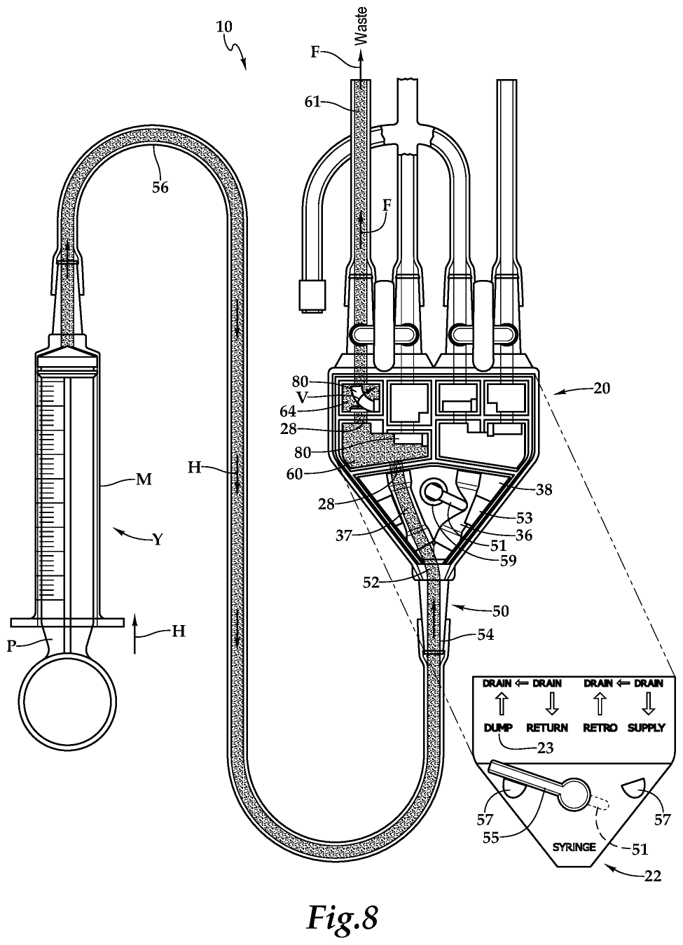

In a fourth and final step of this mode, revealed in FIG. 8, the plunger P of the syringe Y is depressed (along arrow H). This causes the waste fluid to be advanced along the line 56 through the pressure control port 50, along the second tube 60, through the second chamber 60, and then through the post chamber 64 to the discharge port 75 (along arrow F). This discharge port 75 can be coupled to discharge line 61 which leads to a waste fluid collector. The fourth step occurs with the second clip 76 in place closing off the second bypass tube 74.

In a second mode of operation, rather than inducing flow for irrigation of the bladder, flow occurs under passive gravity forces, depicted in FIG. 9. Initially, the first clip 46 and second clip 76 are removed so that the first bypass tube 44 and second bypass tube 74 are both open. The irrigation fluid source is elevated above the tool 10, so that gravity flow occurs from the area of fluid supply along arrow C to the intake port 40. The flow then passes through the bypass ports 42 along the first bypass tube 44 (along arrow K) to the supply port 45. The bladder of the patient is located at a similar elevation as the tool 10, so the gravity pressure from the elevated source of irrigation fluid causes flow from the supply port 45 along arrow D to the solution lumen S of the triple lumen catheter T and into the bladder. The output lumen O of the triple lumen catheter T leads to the drain port 70. Because the bypass port 72 and second bypass tube 74 is open and active, flow of waste fluid from the bladder passes along arrow E to the drain port 70, and then along the bypass tube 74 (along arrow L) to the discharge line 61 coupled to the discharge port 75, and routed on to the waste fluid collector or other discharge location (along arrow F). The end of the discharge line 61 opposite discharge port 75 and a waste fluid collector are preferably located below the tool 10 to further encourage gravity forces to enable natural flow into and out of the bladder for irrigation thereof. This passive second mode of operation could be encouraged to occur slightly faster such as by further elevating the source of irrigation fluid, or squeezing the source of irrigation fluid, if it is an irrigation liquid within a bag, such as hanging on an IV pole. Other devices such as infusion pumps could be utilized to apply a slight pressure while still keeping the tool 10 in bypass mode.

While in this second mode of operation, a medical professional can still utilize the pathway selector to make either the supply pathway active or to make the drain pathway active. The pathway that is active can have its associated clip placed upon the associated bypass tube 44, 74 and one or more cycles of retracting and depressing the plunger P of the strange way I can occur to provide some pulses of fluid flow through the bladder of the patient. This can allow for more current diagnostic information such as an amount of blood in the waste flow, pain associated with bladder pressurization, and other diagnostic criteria can be evaluated. When a medical professional is not present, the tool 10 can merely operate in a passive mode to slowly provide bladder irrigation for therapeutic benefit without requiring as close of active monitoring.

This disclosure is provided to reveal a preferred embodiment of the invention and a best mode for practicing the invention. Having thus described the invention in this way, it should be apparent that various different modifications can be made to the preferred embodiment without departing from the scope and spirit of this invention disclosure. When embodiments are referred to as “exemplary” or “preferred” this term is meant to indicate one example of the invention, and does not exclude other possible embodiments. When structures are identified as a means to perform a function, the identification is intended to include all structures which can perform the function specified. When structures of this invention are identified as being coupled together, such language should be interpreted broadly to include the structures being coupled directly together or coupled together through intervening structures. Such coupling could be permanent or temporary and either in a rigid fashion or in a fashion which allows pivoting, sliding or other relative motion while still providing some form of attachment, unless specifically restricted.

What is claimed is:

1: A bladder irrigation tool, comprising in combination:
a supply port connectable to a supply lumen of a bladder catheter;
an intake port connectable to a source of irrigation fluid;
a drain port connectable to a drain lumen of a bladder catheter;
a discharge port connectable to a waste fluid collector;
a pressure control port collectible to a pressure and vacuum applicator;
a supply pathway having two ends including a first end and a second end, said supply pathway extending between said supply port and said intake port at said first end and said pressure control port at said second end of said supply pathway;
a drain pathway having two ends including a first end and a second end, said drain pathway extending between said drain port and said discharge port at said first end of said drain pathway and said pressure control port at said second end of said drain pathway; and
a pathway selector selectively blocking one of said supply pathway or said to drain pathway.

2: The tool of claim 1 wherein a first chamber is located at a junction with access to all three of said supply port, said intake port and said first end of said supply pathway.

3: The tool of claim 2 wherein a first check valve is located between said first chamber and said supply port and a second check valve is located between said first chamber and said intake port, said second check valve oriented to prevent flow out of said first chamber and into said intake port and said first check valve oriented to prevent flow out of said supply port and into said first chamber.

4: The tool of claim 2 wherein a second chamber is located at a junction with access to all three of said drain port, said discharge port and said first end of said drain pathway.

5: The tool of claim 4 wherein a first check valve is located between said second chamber and said drain port and a second check valve is located between said second chamber and said discharge port, said second check valve oriented to prevent flow out of said second chamber and into said drain port and said first check valve oriented to prevent flow out of said discharge port and into said second chamber.

6: The tool of claim 5 wherein at least one of said check valves includes a hole passing through a seat, with a flap adjacent to said hole and sized and shaped to pivot to block said hole.

7: The tool of claim 6 wherein said hole includes a protrusion at a perimeter thereof and extending toward said flap, and with said flap including a cut out having a contour complementary to that of said protrusion, said protrusion being frusto-conical in form and said cut out being of a complementary frusto-conical form to said protrusion.

8: The tool of claim 1 wherein a syringe is connected at least indirectly to said pressure control port, with said syringe including a plunger translating within a chamber, with retraction of said plunger drawing a vacuum in at least one of said supply pathway or said drain pathway, and depressing of said plunger adding pressure to at least one of said supply pathway or said drain pathway.

9: The tool of claim 1 wherein at least a portion of said supply pathway is formed of a collapsible conduit, and at least a portion of said drain pathway is formed of a collapsible conduit, said pathway selector located adjacent to said collapsible conduit of said supply pathway and said collapsible conduit of said drain pathway, with said pathway selector configured to impinge and collapse one of said collapsible conduit of said supply pathway or said collapsible conduit of said drain pathway.

10: The tool of claim 9 wherein said pathway selector includes a rotatable shaft located between said collapsible conduit of said supply pathway and said collapsible conduit of said drain pathway, said rotatable shaft including a finger extending radially from a pivot axis of said rotatable shaft, said finger long enough to push against and close either said collapsible conduit of said supply pathway or said collapsible conduit of said drain pathway, when said rotatable shaft is rotated to bring said finger into contact with one of said collapsible conduits.

11: The tool of claim 1 wherein said pathway selector includes a lever which is manually rotatable and points toward indicia visible on a cover of the tool, said indicia that the lever is pointing to identifying said pathway which is left open by sad pathway selector.

12: The tool of claim 1 wherein a clean irrigation fluid bypass extends between said intake port and said supply port without accessing said pressure control port, said clean irrigation fluid bypass being selectively openable to allow flow between said intake port and said supply port without accessing said pressure control port.

13: The tool of claim 1 wherein a waste fluid bypass extends between said drain port and said discharge port without accessing said pressure control port, said waste fluid bypass being selectively openable to allow flow between said drain port and said discharge port without accessing said pressure control port.

14: The tool of claim 13 wherein a clean irrigation fluid bypass extends between said intake port and said supply port without accessing said pressure control port, said clean irrigation fluid bypass being selectively openable to allow flow between said intake port and said supply port without accessing said pressure control port, and wherein said waste fluid bypass and said clean irrigation fluid bypass are each at least partially formed of flexible materials, and selectively including clips thereon to removably pinch closed one or both of said clean irrigation fluid and/or said waste fluid.

15: A method for irrigating a patient's bladder, including the steps of:
connecting a supply port of a bladder irrigation tool to a supply lumen of a bladder catheter;
connecting an intake port of the bladder irrigation tool to a source of irrigation fluid;
connecting a drain port of the bladder irrigation tool to a drain lumen of a bladder catheter;
connecting a discharge port of the bladder irrigation tool to a waste fluid collector;
connecting a pressure control port to a pressure and vacuum applicator of the bladder irrigation tool; and
blocking one of a supply pathway or a drain pathway of the bladder irrigation tool, the supply pathway having two ends including a first end and a second end, the supply pathway extending between the supply port and the intake port at the first end and the pressure control port at the second end, and the drain pathway having two ends including a first end and a second end, the drain pathway extending between the drain port and the discharge port at the first end and the pressure control port at the second end.

16: The method of claim 15 including the further step of applying vacuum to draw irrigation fluid in through the intake port, followed by applying pressure to advance the irrigation fluid out of the supply port to the supply lumen of the bladder catheter.

17: The method of claim 15 including the further step of applying vacuum to draw waste fluid in through the drain port from the drain lumen of the bladder catheter, followed by applying pressure to advance the waste fluid out of the discharge port to the waste fluid collector.

18: The method of claim 15 wherein said blocking step includes at least a portion of the supply pathway being collapsible and a portion of the drain pathway being collapsible, and with a pathway selector movable to block by collapsing either the supply pathway or the drain pathway.

19: The method of claim 15 wherein said applying a vacuum step and said applying pressure step are each provided by a syringe coupled to the pressure control port, with retracting of a plunger of the syringe drawing a vacuum on at least one of the supply pathway or the drain pathway, and depressing of the plunger of the syringe applying pressure to at least one of the supply pathway or the drain pathway.

20: The method of claim 15 including the further step of bypassing at least one of the supply pathway or the drain pathway by providing an openable bypass conduit between said intake port and said supply port and an openable bypass conduit between said drain port and said discharge port.
